(19) United States
(12) Patent Application Publication
(10) Pub. No.: US 2026/0216131 A1
(43) Pub. Date: Jul. 30, 2026

(54) METHODS OF TREATING BREAST HYPERPLASIA USING TEZACAFTOR

(71) Applicant: Jiangsu Bioscene Pharmaceutical Co., Ltd., Changzhou (CN)

(72) Inventors: Jian HOU, Changzhou (CN); Zhengtao ZONG, Changzhou (CN); Ningpu YU, Changzhou (CN); Zaixin CHEN, Changzhou (CN)

(73) Assignee: Jiangsu Bioscene Pharmaceutical Co., Ltd., Changzhou (CN)

(21) Appl. No.: 19/577,557

(22) Filed: Mar. 25, 2026

Related U.S. Application Data
(63) Continuation of application No. PCT/CN2024/116859, filed on Sep. 4, 2024.

(30) Foreign Application Priority Data
Sep. 26, 2023 (CN) 202311247766.5

(51) Int. Cl. A61K 31/404; A61K 9/00; A61K 9/16; A61K 9/20; A61K 9/28; A61P 43/00

(52) U.S. Cl. CPC A61K 31/404; A61K 9/0019; A61K 9/0053; A61K 9/1652; A61K 9/2013; A61K 9/2054; A61K 9/28; A61P 43/00

(57) ABSTRACT

The present invention relates to a method for treating breast hyperplasia in a subject in need thereof, comprising administering to the subject tezacaftor, or a pharmaceutically acceptable salt, solvate, or hydrate thereof. Non-clinical pharmacodynamic studies demonstrated that, in a rat model of breast hyperplasia, tezacaftor significantly reduced nipple diameter, nipple height, and uterine index, alleviated breast hyperplasia, and improved serum E2 and PROG levels. These results indicate that tezacaftor is useful for treating breast hyperplasia.

### TECHNICAL FIELD OF THE INVENTION

The present invention relates to the field of biopharmaceuticals, and more particularly to a method for treating breast hyperplasia, comprising administering tezacaftor, or a pharmaceutically acceptable salt thereof, a solvate of any of the foregoing, or a hydrate of any of the foregoing.

### BACKGROUND OF THE INVENTION

Breast hyperplasia is a benign breast disease resulting from abnormalities in the normal developmental and involutional processes of the breast. Essentially, it is a disorder of the normal breast architecture caused by varying degrees of hyperplasia of the breast parenchyma and stroma, together with incomplete involution. Its pathological manifestations are diverse and complex, and therefore its clinical nomenclature has not been uniform.

Epidemiological data show that breast hyperplasia is one of the most common breast diseases in women and has the highest incidence among breast diseases. Some studies have shown that, in recent years, the incidence of this disease has increased year by year, and the age of onset has tended to become younger. According to expert reports, 70% to 80% of women have breast hyperplasia to varying degrees.

At present, the etiology and pathogenesis of breast hyperplasia remain unclear. It is generally accepted that endocrine disorder is a major cause thereof, and the associated clinical manifestations are considered external manifestations of hormonal imbalance in the body. Such factors include: (1) an imbalance in the ratio of estrogen to progesterone, resulting in excessive proliferation and incomplete involution of the mammary glands; (2) abnormal distribution and expression of estrogen receptors in various parts of the breast; and (3) elevated prolactin levels, which indirectly affect breast development and interfere with the function of the hypothalamic-pituitary-gonadal axis. In addition, because the disease often persists for a prolonged period and is prone to recurrence, it seriously affects the physical and mental health of many women.

Currently, treatment options for breast hyperplasia are limited. Certain therapeutic agents have been used in clinical practice; however, their efficacy may be unsatisfactory in some patients, and some agents may be associated with undesirable adverse effects. Accordingly, there remains a need for improved therapeutic agents for treating breast hyperplasia, particularly chemical drugs with good efficacy and acceptable safety profiles.

### SUMMARY OF THE INVENTION

The present invention provides a method for treating breast hyperplasia, comprising administering tezacaftor, or a pharmaceutically acceptable salt thereof, a solvate of tezacaftor, a hydrate of tezacaftor, a solvate of the pharmaceutically acceptable salt, or a hydrate of the pharmaceutically acceptable salt. The present invention further provides a pharmaceutical composition for treating breast hyperplasia.

In some embodiments, the present invention provides a method for treating breast hyperplasia, comprising administering a compound of Formula I, or a pharmaceutically acceptable salt thereof, a solvate of tezacaftor, a hydrate of tezacaftor, a solvate of the pharmaceutically acceptable salt, or a hydrate of the pharmaceutically acceptable salt. In some embodiments, the compound of Formula I is tezacaftor.

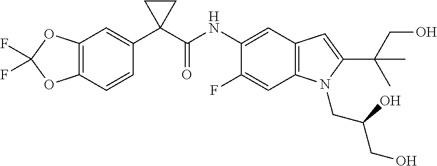 [SMILES: CC(C)(CO)c1cc2cc(NC(=O)C3(c4ccc5c(c4)OC(F)(F)O5)CC3)c(F)cc2n1C[C@@H](O)CO]

In some embodiments, the solvate of tezacaftor, or the solvate of a pharmaceutically acceptable salt thereof, is an o-xylene solvate.

The present invention further relates to a pharmaceutical composition comprising a compound of Formula I, a pharmaceutically acceptable salt thereof, a solvate of any of the foregoing, or a hydrate of any of the foregoing. The pharmaceutical composition may further comprise one or more pharmaceutically acceptable carriers or excipients. In certain embodiments, the pharmaceutical composition is formulated as a solid dosage form, a liquid dosage form, an oral formulation, or a parenteral formulation.

The pharmaceutical composition may be administered by any suitable route, including oral administration or parenteral administration, such as subcutaneous, intravenous, or intramuscular administration. In certain embodiments, oral administration is preferred.

For oral administration, the compound of Formula I, a pharmaceutically acceptable salt thereof, a solvate of any of the foregoing, or a hydrate of any of the foregoing may be formulated in any pharmaceutically acceptable oral dosage form, including but not limited to tablets, capsules, aqueous solutions, and suspensions. In some embodiments, tablets may contain carriers or excipients such as lactose, corn starch, microcrystalline cellulose, and sodium carboxymethyl cellulose, and may optionally further contain a lubricant such as magnesium stearate. Capsules may contain diluents such as lactose and dried corn starch. Suspensions may be prepared by combining the active ingredient with suitable emulsifying agents and suspending agents. If desired, any of the foregoing oral dosage forms may further comprise sweetening agents, flavoring agents, or coloring agents.

For parenteral administration, the compound of Formula I, a pharmaceutically acceptable salt thereof, a solvate of any of the foregoing, or a hydrate of any of the foregoing may be formulated as an injectable solution or a powder for injection. Suitable carriers may include, without limitation, water for injection, bulking agents, pH-adjusting agents, antioxidants, and supporting agents.

Non-clinical pharmacodynamic studies showed that tezacaftor, a compound of Formula I, significantly reduced nipple diameter, nipple height, and uterine index in a rat model of breast hyperplasia. Tezacaftor also alleviated breast hyperplasia and improved serum E2 and PROG levels in the model animals. These findings suggest that tezacaftor may be effective in the treatment of breast hyperplasia. In some embodiments, the subject in need of treatment is a mammal, such as a human, a canine, or a rodent.

### BRIEF DESCRIPTION OF DRAWINGS

FIG. 1 shows the nipple size of rats in each group after 30 days of drug treatment.

FIG. 2 shows the ovarian index and uterine index of rats in each group after 30 days of drug treatment.

FIG. 3 shows the serum estrogen and progesterone levels in rats in each group after 30 days of drug treatment.

FIG. 4 shows stained sections of breast tissue from rats in each group after 30 days of drug treatment.

### DETAILED DESCRIPTION OF THE INVENTION

The present invention is further described in detail below with reference to the Embodiments. However, the present invention is not limited thereto.

### Embodiment 1: Pharmacodynamic Study of Tezacaftor in a Rat Model of Breast Hyperplasia

Study Design: Thirty-two female SD rats, aged 7-8 weeks and weighing 200-250 g, were used. The rats were randomly divided into four groups: a normal control group, a model group, a tezacaftor treatment group (hereinafter referred to as the “treatment group”), and a tamoxifen treatment group (hereinafter referred to as the “positive control group”).

Before administration of the test drugs, breast hyperplasia was induced in the rats of the model group, the treatment group, and the positive control group. Specifically, estradiol benzoate injection was intramuscularly administered to the inner thigh of each rat at a dose of 0.5 mg/kg/day for 25 consecutive days. Starting on day 26, progesterone injection was intramuscularly administered to the inner thigh at a dose of 5 mg/kg/day for 5 consecutive days. The total modeling period was 30 days.

Normal control group: Normal saline in an equal volume was intramuscularly administered to the inner side of the hind limb of each rat once daily for 30 consecutive days, with the left and right hind limbs injected alternately. Beginning on day 31, normal saline was administered by gavage for 30 days.

Model group: Beginning on day 31, the vehicle was administered by gavage for 30 consecutive days. Samples were collected 0.5 hour after the last administration.

Treatment group: Beginning on day 31, tezacaftor was administered by gavage at a dose of 40 mg/kg/day for 30 consecutive days. Samples were collected 0.5 hour after the last administration.

Positive control group: Beginning on day 31, tamoxifen was administered by gavage at a dose of 4 mg/kg/day for 30 consecutive days. Samples were collected 0.5 hour after the last administration.

Evaluation indices: (1) nipple diameter and height; (2) ovarian index and uterine index; (3) changes in serum E2 and PROG levels; and (4) the number of mammary lobular acini, the acinar lumen diameter, and the duct diameter in rats.

### Embodiment 2: Evaluation of Nipple Size in Rats with Breast Hyperplasia Treated with Tezacaftor

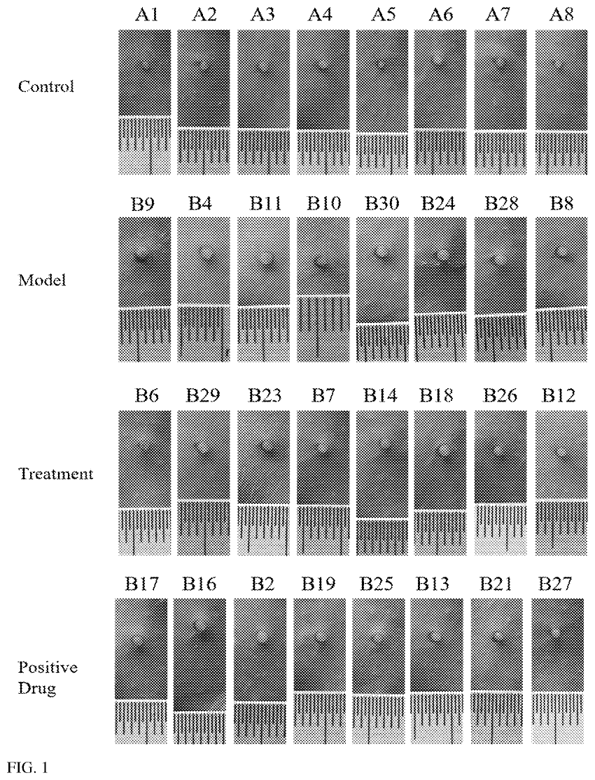

In the study of Embodiment 1, after 30 days of administration, the diameter and height of the nipples of the rats were measured. Representative images showing nipple size in the rats of each group are presented in FIG. 1. The data for nipple diameter and height in each group are shown in Tables 1 and 2, respectively.

The results showed that, as compared with the model group, the treatment group and the positive control group exhibited decreases in both nipple diameter and nipple height, and the differences were statistically significant.

TABLE 1
Nipple Diameter of Rats in Each Group at Different Time Points of Treatment

| | Normal | | | Positive |
|---|---|---|---|---|
| Group | Control Group | Model Group | Treatment Group | Control Group |
| Day 0 of treatment | 1.08±0.04 | 1.63 ± 0.25 | 1.63 ± 0.14 | 1.63 ± 0.12 |
| Day 15 of treatment | 1.03 ± 0.06 | 1.42 ± 0.10 | 1.24 ± 0.13 | 1.28 ± 0.14 |
| Day 30 of treatment | 1.03 ± 0.05 | 1.32 ± 0.12 | 1.12 ± 0.08 ** | 1.11 ± 0.10*** |

TABLE 2
Nipple Height (mm) of Rats in Each Group at Different Time Points of Treatment

| | Normal | | | Positive |
|---|---|---|---|---|
| Group | Control Group | Model Group | Treatment Group | Control Group |
| Day 0 of treatment | 1.26 ± 0.08 | 1.89 ± 0.16 | 2.03 ± 0.12 | 1.94 ± 0.16 |
| Day 15 of treatment | 1.27 ± 0.06 | 1.92 ± 0.24 | 1.73 ± 0.21 | 1.70 ± 0.17 |
| Day 30 of treatment | 1.24 ± 0.12 | 1.82 ± 0.19 | 1.61 ± 0.13* | 1.56 ± 0.12** |

**Data were analyzed by one-way ANOVA. Compared with the model group, *P < 0.05, **P < 0.01, and ***P < 0.001.

### Embodiment 3: Evaluation of Ovarian Index and Uterine Index in Rats with Breast Hyperplasia Treated with Tezacaftor

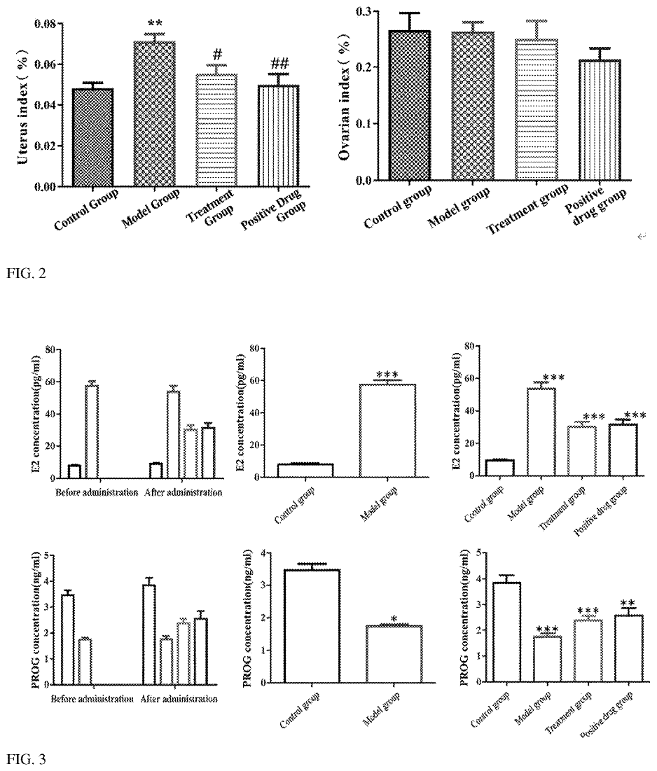

In the study of Embodiment 1, after 30 days of administration, the uterus weight, ovary weight, and body weight of the rats were measured. The uterine index was calculated as uterine weight/body weight, and the ovarian index was calculated as ovary weight/body weight. A comparison of the ovarian index and uterine index among the different groups is shown in FIG. 2, and the corresponding data are presented in Table 3.

The results showed that, as compared with the model group, the treatment group and the positive control group exhibited decreases in both ovarian index and uterine index, and the differences were statistically significant.

TABLE 3
Uterine Index and Ovarian Index of Rats in Each Group

| | Normal | | | Positive |
|---|---|---|---|---|
| Group | Control Group | Model Group | Treatment Group | Control Group |
| Uterine index (%) | 0.048 ± 0.008 | 0.071 ± 0.011 | 0.055 ± 0.012* | 0.050 ± 0.015** |
| Ovarian index (%) | 0.210 ± 0.085 | 0.203 ± 0.047 | 0.186 ± 0.088 | 0.145 ± 0.052 |

**Data were analyzed by one-way ANOVA. Compared with the model group, *P < 0.05, **P < 0.01, and ***P < 0.001.

### Embodiment 4: Evaluation of Serum E2 and PROG Levels in Rats with Breast Hyperplasia Treated with Tezacaftor

In the study of Embodiment 1, after 30 days of administration, serum E2 and PROG levels in rats of each group were measured using assay kits. A comparison of these two parameters among the different groups is shown in FIG. 3.

The results show ed that, as compared with the model group, the treatment group and the positive control group exhibited significantly decreased serum E2 levels and significantly increased serum PROG levels, and both differences were statistically significant.

### Embodiment 5: Histopathological Evaluation of Mammary Glands in Rats with Breast Hyperplasia Treated with Tezacaftor

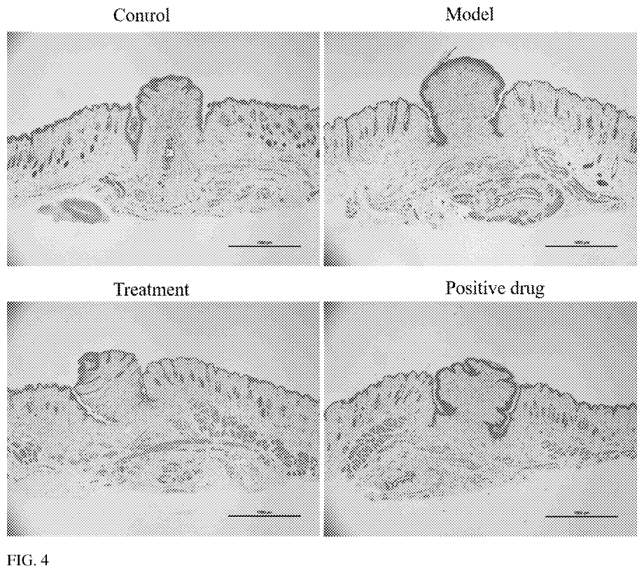

In the study of Embodiment 1, after 30 days of administration, mammary gland tissues from rats in each group were collected, dehydrated, embedded, and sectioned. The sections were subjected to H&E staining and pathological analysis. Representative H&E staining results of the mammary glands from each group are shown in FIG. 4. The pathological examination scoring results are presented in Table 4.

The results showed that no abnormality was observed in the mammary glands of the normal control group. In the model group, obvious breast hyperplasia was observed, as evidenced by an increased number of lobules and acini, dilation of ducts and acini, abundant secretions, and marked stromal hyperplasia. As compared with the model group, breast hyperplasia in the positive control group and the treatment group was alleviated.

TABLE 4
Histopathological Examination Scores

| | Normal | | Treatment | Positive |
|---|---|---|---|---|
| Animal No. | Control Group | Model Group | Group | Control Group |
| 1 | 0 | 3 | 2 | 2 |
| 2 | 0 | 2 | 2 | 1 |
| 3 | 0 | 2 | 1 | 2 |
| 4 | 0 | 2 | 1 | 1 |
| 5 | 0 | 2 | 1 | 0 |
| 6 | 0 | 1 | 1 | 1 |
| 7 | 0 | 1 | 2 | 2 |
| 8 | 0 | 2 | 2 | 1 |

Note:
All morphological changes were scored according to severity as 1, 2, 3, or 4, representing very slight, mild, moderate, and severe, respectively. Absence of a lesion was scored as 0, and missing data were indicated as “N/A.”

### Embodiment 6: Comprehensive Evaluation of the Therapeutic Efficacy of Tezacaftor in Rats with Breast Hyperplasia

Based on the results and evaluation of the foregoing indicators, administration of tezacaftor to rats with hormone-induced breast hyperplasia significantly reduced nipple diameter, nipple height, and uterine index, thereby alleviating breast hyperplasia. In addition, the serum levels of E2 and PROG were significantly improved. All of the above differences were statistically significant, indicating that tezacaftor is useful for the treatment of breast hyperplasia.

### Embodiment 7: Preparation of a Pharmaceutical Composition Containing Tezacaftor—Oral Tablet Formulation

TABLE 5

| Ingredient | Amount | Function |
|---|---|---|
| Tezacaftor | 50 mg | Active ingredient |
| Hydroxypropyl cellulose | 7.5 mg | Binder |
| Sodium lauryl sulfate | 2.5 mg | Solubilizer |
| Microcrystalline cellulose | 117.5 mg | Filler |
| Croscarmellose sodium | 20 mg | Disintegrant |
| Magnesium stearate | 2.5 mg | Lubricant |
| Tablet core | 200 mg | |
| Film-coating powder | 8 mg | Coating material |
| Tablet weight | 208 mg | |

### Preparation Process:

The prescribed amounts of tezacaftor and microcrystalline cellulose were weighed and mixed in a wet granulator for 5 minutes.

The prescribed amount of sodium lauryl sulfate was added to the above powder mixture and mixed for 5 minutes.

The prescribed amount of hydroxypropyl cellulose was weighed and dissolved in 150 mL of water to prepare a 5% hydroxypropyl cellulose solution.

The hydroxypropyl cellulose solution was added to the powder mixture, and granulation was performed in a wet granulator for 10 minutes.

The resulting granules were dried in a fluidized-bed dryer for 20 minutes.

The dried granules were mixed with the prescribed amounts of croscarmellose sodium and magnesium stearate in a blender for 5 minutes.

The resulting powder mixture was compressed into uncoated tablets, each having a tablet core weight of 200 mg.

The uncoated tablets were film-coated with the coating material, and coating was stopped after a 4% weight gain was achieved, thereby obtaining the final product.

### Embodiment 8: Preparation of a Pharmaceutical Composition Containing Tezacaftor—Lyophilized Powder for Injection

TABLE 6

| Ingredient | Amount |
|---|---|
| Tezacaftor monohydrate | 50 mg (calculated as |
| | tezacaftor) |
| Sulfobutyl beta-cyclodextrin | 1500 mg |
| Methanol | 3 mL |
| Purified water | 11 mL |

### Preparation Process:

The prescribed amount of tezacaftor monohydrate was weighed, dissolved in 1 mL of methanol, and stirred at 450 rpm for 5 minutes.

The prescribed amount of sulfobutyl beta-cyclodextrin was weighed and dissolved in 2 mL of methanol and 1 mL of water until a clear solution was obtained.

The above two solutions were combined and stirred at 450 rpm for 2 hours, followed by sterilization through a 0.22 μm filter membrane.

The resulting solution was vacuum-dried in a 50° C. water bath at 60 rpm to obtain a solid. The above solid was reconstituted with 10 mL of water for injection, followed by lyophilization in a freeze dryer to obtain the final product.

All references cited herein are incorporated by reference in their entirety, as if each reference were individually and specifically incorporated by reference.

1. A method of treating breast hyperplasia in a subject in need thereof, comprising administering to the subject an effective amount of tezacaftor or a pharmaceutically acceptable salt thereof, a solvate of any of the foregoing, or a hydrate of any of the foregoing.

2. The method of claim 1, wherein the subject is a human.

3. The method of claim 1, wherein the subject is a canine, a rodent, or another mammal.

4. The method of claim 1, wherein the breast hyperplasia is benign breast hyperplasia.

5. The method of claim 1, wherein the tezacaftor is administered orally.

6. The method of claim 1, wherein the tezacaftor is administered parenterally.

7. The method of claim 1, wherein the tezacaftor is administered in a pharmaceutical composition further comprising a pharmaceutically acceptable carrier or excipient.

8. The method of claim 7, wherein the pharmaceutical composition is a solid dosage form or a liquid dosage form.

9. The method of claim 8, wherein the pharmaceutical composition is a tablet, a capsule, an aqueous solution, or a suspension.

10. The method of claim 8, wherein the pharmaceutical composition is an injection or a lyophilized powder for injection.

11. The method of claim 1, wherein the administered compound is tezacaftor monohydrate.

12. The method of claim 1, wherein the administered compound is tezacaftor o-xylene solvate.

13. A pharmaceutical composition for use in treating breast hyperplasia comprising tezacaftor or a pharmaceutically acceptable salt thereof, and a pharmaceutically acceptable carrier or excipient.

14. The pharmaceutical composition of claim 13, wherein the pharmaceutical composition is an oral formulation or a parenteral formulation.

15. The pharmaceutical composition of claim 14, wherein the oral formulation is a tablet, a capsule, an aqueous solution, or a suspension.
